(19) United States
(12) Patent Application Publication
(10) Pub. No.: US 2007/0082864 A1
(43) Pub. Date: Apr. 12, 2007

(54) Method for inducing a specific rnai activity in cells and nucleic acids for carrying out said method

(76) Inventors: Annick Harel-Bellan, Paris (FR); Slimane Ait-Si-Ali, Villejuif (FR); Monsef Benkirane, Quissac (FR)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 10/576,390

(22) PCT Filed: Oct. 19, 2004
(86) PCT No.: PCT/FR04/02673

§ 371 Date: May 31, 2006

(30) Foreign Application Priority Data
Oct. 20, 2003 (FR) 0312250

(51) Int. Cl. A61K 48/00; C07H 21/04; C12N 15/86

(52) U.S. Cl. 514/44; 435/456; 435/325; 536/23.2

(57) ABSTRACT

A method for inducing activity of a specific RNAi into eukaryote cells in which the TAT protein and a nucleic acid coding for a nucleotide sequence comprising the sense and antisense sequences of an RNAi of interest are introduced, which sense and antisense sequences are separated by a separating nucleotide sequence coding for a sequence comprising at least the minimal TAR sequence recognized by the TAT protein, which transcribed separating sequence and the TAT protein form a complex that inhibits the activity of the RNAi of interest, and the activity of the RNAi is induced by withdrawing the TAT protein.

### RELATED APPLICATION

This is a §371 of International Application No. PCT/FR2004/002673, with an international filing date of Oct. 19, 2004 (WO 2005/040385, published May 6, 2005), which is based on French Patent Application No. 03/12250, filed Oct. 20, 2003.

### TECHNICAL FIELD

This disclosure relates to biology and, more particularly, to preparation of double-stranded oligonucleotides for use in an RNA interference process (RNAi) to induce degradation of a target RNA.

### BACKGROUND

Interference RNA, also designated “RNAi”, or even co-suppression, has been detected in plants, where it was observed that the introduction of a long, double-stranded RNA corresponding to a gene induces specific and efficacious repression of the expression of the targeted gene. The mechanism of this interference comprises degradation of the double-stranded RNA into short duplexes of oligonucleotides of approximately 20 to 22 nucleotides called siRNA's.

Interference RNA has now been applied to mammals for specifically inhibiting expression of genes for applications in functional genetics. In fact, siRNA's permit identification of the function of genes detected by sequencing of the human genome either in cellular culture models or in animal models and, in particular, in the mouse. Interference RNA is also useful in the therapeutic area for the treatment or prevention of cancers, infectious diseases and, more generally, of diseases involving a heterologous or homologous mutated gene (S. M. Elbashir, J. Harborth, W. Lendeckel, A. Yalcin, K. Weber and T. Tuschi (2001a), “Duplexes of 21-Nucleotide RNA's Mediate RNA Interference in Cultured Mammalian Cells”, Nature 411, 494-498; S. M. Elbashir, J. Martinez, A. Patkaniowska, W. Lendeckel and T. Tuschi (2001b), “Functional Anatomy of siRNA's for Mediating Efficient RNAi in Drosophila melanogaster Embryo Lysate”, Embo J. 20 6877-6888).

siRNA's are short sequences of double-stranded RNA that can be introduced into cells in the form of synthetic oligonucleotides or in the form of vectors permitting expression of these siRNA's. In this latter approach, siRNA is synthesized in the form of a precursor that is a shRNA (short hairpin RNAi), formed by a “rod loop” in which the rod represents the siRNA and the loop any sequence that will be degraded by the cellular RNAase's, which will give rise to the mature siRNA.

Implementation of siRNA expression vectors has numerous advantages, in particular, for applications of functional genetics. It allows double-stranded RNA to be expressed in a stable manner in cells and therefore to more readily inhibit expression of proteins with a long half-life. In fact, synthetic siRNA's have a half-life of 3 days in mammalian cells. Furthermore, siRNA's are diluted in the course of cellular divisions.

It also permits long-term effects to be analyzed. On the other hand, it requires establishment of lines expressing the construction in a stable manner, which presents several disadvantages. In particular, it is necessary to compare stable lines with each other, which is generally difficult to interpret because the cellular lines drift. On the other hand, it is impossible to study the proteins indispensable for the cell since their inhibition will block proliferation of the cells and will thus prevent establishment of the stable line. It is therefore indispensable to be able to induce the activity of siRNA. The term “induce the activity of siRNA” denotes being able to block and unblock its activity substantially at will. The term “activity of siRNA” denotes its capacity to recognize and induce degradation of its target RNA.

The prior art describes a vector that permits the stable and inducible expression of siRNA. That system is based on inhibition of the transcription of the precursor of siRNA. In fact, the vector, derived from the pSUPER vector (T. R. Brummelkamp et al, www.schencexpress.org /Mar. 21, 2002/page 1/10. 1126/science.1068999), contains a known sequence, “the tetracycline operator” (“TET operator”), positioned between the promoter directing transcription of the siRNA and the sequence coding that siRNA. In the presence of the protein “TET repressor,” the latter fixes itself on the “TET operator” and thus blocks transcription of the siRNA. In the presence of doxycycline, the latter inhibits fixation of the “TET repressor” on the “TET operator” and thus permits transcription of the siRNA. This expression system engenders a relatively elevated background amount of activity;noise.

Moreover, the inducible systems based on the promoters functions very poorly in viral vectors because the viral promoters take precedence over the inducible promoters and the background activity/noise is very elevated.

### SUMMARY

This invention relates to a method for inducing activity of an RNAi in cells including introducing TAT protein and a nucleic acid coding for sense and antisense sequences of an RNAi of interest into eukaryote cells, which sense and antisense sequences are separated by a nucleotide separating sequence including at least SEQ ID No. 1, coding for at least one nucleotide sequence contained in a TAR sequence for a time sufficient to be recognized by the TAT protein under conditions in which the nucleic acid is transcribed in RNA so that the transcribed separating sequence and the TAT protein form a complex inhibiting the activity of the RNAi of interest, and withdrawing the TAT protein to induce activity of the RNA.

This invention also relates to a cell or a line of cells transfected by the nucleic acid.

This invention further relates to a pharmaceutical composition including at least one nucleic acid and, optionally, a compatible excipient.

This invention still further relates to a pharmaceutical composition including a cell or line of cells and, optionally, a compatible excipient.

### BRIEF DESCRIPTION OF THE DRAWINGS

FIG. 1 is a Western blot that used a total extract with an antibody directed against the GFP or cellular tubulin to evaluate the quantity of proteins.

### DETAILED DESCRIPTION

The method described herein remedies these disadvantages by providing a system that permits the activity of a siRNA to be induced substantially at will. This is achieved by a protein couple TAT-sequence RNA TAR to block the activity of a siRNA in mammalian cells.

The principle is based on the interaction of TAT/TAR. In fact, the siRNA is recognized in the cell by a protein complex called “RISC complex” (RNAi-Induced Silencing Complex) that causes it to undergo a maturation stage and uses it as a guide to recognize and degrade the target mRNA. The TAT protein can be used to block the latter stage.

The TAT protein of VIH plays a crucial part in viral replication. In fact, TAT regulates, as a transcription factor, replication speed of the virus. TAT is a secreted protein capable of diffusing into non-infected cells and preparing an environment propitious for viral infection. The TAT protein exists in two forms (71 and 101 and amino acids) coded by two exons separated by the env gene. TAT exercises its function as activator of the promoter of VIH by binding to the RNA TAR sequence present at 5′ of all the RNA's of the VIH.

Linking TAT and its transcriptional cellular co-activators to RNA TAR stimulates elongation of the transcription by augmenting the processivity of RNA polymerase II.

The presence of double-stranded RNA's in a cell represents a signal: It involves an RNaseIII called Dicer. This ribonuclease cleaves the long, double-stranded RNA's into small fragments of double-stranded RNA, the siRNA's, with a precise size and structure, in general 21 to 23 nucleotides. The duplex siRNA's then become associated with proteins to form a complex called RISC (for “RNAi-Induced Silencing Complex”). RISC is therefore a ribonucleoprotein complex containing a molecule of siRNA associated with proteins belonging to the Argonaut family. RISC guides the small siRNA's by virtue of the 5′ phosphate end of a simple-stranded siRNA for the specific recognition of the target messenger RNA and catalyses its cleavage. This reaction takes place in the cytoplasm. This terminates in the inhibition of the translation and thus in the inhibition of the expression of the target gene.

The loop of the precursor of the siRNA (shRNA) normally constituted by any sequence, is replaced by a nucleotide sequence coding for at least one nucleotide sequence contained in the TAR sequence, minimal and sufficient for being recognized by the TAT protein.

This sequence is SEQ ID No. 1 and is composed of 11 nucleotides and has the following DNA banding pattern: ATCTGAGCTCT (or AUCUGAGCUCU in its RNA form).

It is also possible to use a sequence coding for the entire wild or mutated TAR sequence or any fragment of it containing this minimal, non-mutated nucleotide sequence described above. This sequence (DNA or RNA) is named elsewhere in the separate sequence text. The separate sequence can be natural or synthetic.

Thus, in the presence of the TAT protein, the latter interacts with the TAR loop on the shRNA, and the interaction of the siRNA protein maturation complex (RISC complex) cannot be realized and the siRNA does not undergo the maturation stages induced by this complex and, therefore, remains inactive, that is to say, in the shRNA form.

In the absence of the TAT protein, the TAR loop of shRNA is degraded and the resulting siRNA is assessable to the RISC maturation complex and the degradation process of the target RNA by the RNAi can thus be carried out.

Therefore, the method for inducing the activity of an RNAi in cells includes:
The TAT protein and a nucleic acid coding for the sense and antisense sequences of an RNAi of interest are introduced into eukaryote cells, which sense and antisense sequences are separated by a nucleotide sequence comprising at least SEQ I No. 1, coding for at least one nucleotide sequence contained in the TAR sequence, minimal and sufficient for being recognized by the TAT protein under conditions in which the nucleic acid is transcribed in RNA, which transcribed separating sequence and the TAT protein form a complex inhibiting the activity of the RNAi of interest;
The activity of the RNAi is induced by withdrawing the TAT protein.

The separating sequence can comprise any nucleotide sequence provided that it comprises at least SEQ ID No. 1. The separating sequence is advantageously constituted a sequence coding for the complete TAR sequence, wild or mutated, or by a fragment of the latter comprising SEQ ID No. 1.

The nucleic acid coding for the sequences may comprise the sense and antisense sequences of an RNAi of interest separated by the separating sequence described above can be introduced into the cell in the form of an expression vector, particularly in the form of a plasmid or a viral vector.

In that instance, the RNAi of interest is transcribed from the vector coding for shRNA that gives rise to the siRNA after cleavage of the single-stranded separating sequence. Thus, the vector comprises at least one nucleotide sequence coding for the sense and antisense sequences of the siRNA separated by a nucleotide sequence comprising at least SEQ ID No. 1 under the control of a transcription promoter.

The nucleic acid described above permits implementation of the method for induction of the activity of an RNAi in eukaryote cells.

Moreover, the vector can comprise an antibiotic resistance gene. The antibiotic resistance gene may be a neomycin resistance gene, for example.

The presence of an antibiotic (e.g. neomycin) resistance gene permits the transfected cells to be selected.

When the cells are cells in culture the TAT protein can be introduced into these cells by a simple culturing of the cells in an environment comprising the TAT protein. In fact, one advantage resides in the fact that the TAT protein penetrates substantially spontaneously into the cells when they are cultivated in a culture environment containing the TAT protein. The action of siRNA comprising the TAR separating sequence as previously described can therefore be readily blocked by cultivating the cells containing the siRNA in a culture environment containing the TAT protein and to stimulate the activity of the RNAi by cultivating these cells in a culture environment without TAT protein, which results in degradation of the target RNA by the RNAi.

Under these conditions, the culture environment can contain the TAT protein at a concentration comprised between about 0.1 μg/ml and about 1.5 μg/ml and very preferentially about 1 μg/ml.

The TAT protein can also be introduced into the cell in the form of an inducible expression vector comprising a nucleotide sequence coding for the TAT protein. In that instance, the expression of the protein can be induced, which has as a consequence the inhibiting the activity of the RNAi and therefore blocking degradation of the target RNA by the RNAi. Degradation of the target RNA by the RNAi can then be induced when the synthesis of the TAT protein is itself blocked.

The cells transfected with the nucleic acid may be mammalian cells. The method applies to transfection of cells in culture as well as directly in the animal.

The method permits genes, especially human genes, to be analyzed in a reliable manner from a functional point of view in cells in culture or in animals, especially in mice.

The method also relates to cells or a cell line in which a nucleic acid as previously described was introduced.

The method also relates to animals comprising cells in which the nucleic acid as previously described was introduced.

Finally, this disclosure includes compositions, especially pharmaceutical compositions comprising as an active substance at least one nucleic acid as previously described or cells containing the nucleic acid such as previously described, optionally associated in the composition with a compatible excipient.

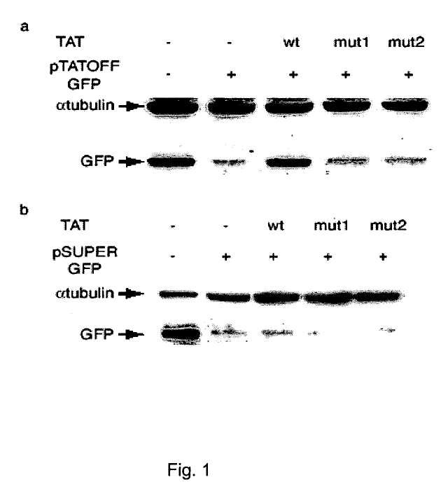

Other advantages and features of the method and composition will appear from the following examples in which reference is made to the attached drawing in which FIG. 1 represents the inhibition of the GFP marker by RNAi.

### EXAMPLE 1

### Construction of the Plasmid pTATOF-siRNAGFP Coding for an RNAi Whose Target is the Messenger RNA Coding for Green Fluorescent Protein (GFP)

This plasmid is constructed from the pSUPER plasmid, permitting the constitutive expression of siRNA and described by Brummelkamp et al.

The DNA sequence (synthetic DNA oligonucleotide synthesized in a hand-tailored manner) containing in order the sequences SEQ ID No. 2 (coding for the sense siRNA)—SEQ ID No. 1 (coding for the separating sequence)—SEQ ID No. 3 (coding for the antisense siRNA) is introduced immediately upstream from the H1 promoter of the pSuper plasmids at sites Bgl II in 5′, and Hind III in 3′. This thus yields the expression vector pTATOF-siRNAGFP.

(SEQ ID NO. 2) coding for the sense siRNA:
5′ GCAAGCTGACCCTGAAGTTC 3′
3′ CGTTCGACTGGGACTTCAAG 5′
Sequence coding for the separating sequence

(SEQ ID No. 1)

5′ ATCTGAGCTCT 3′
3′ TAGACTCGAGA 5′
(SEQ ID NO. 3) coding for the antisense siRNA:
5′ GAACTTCAGGGTCAGCTTGC 3′
3′ CTTGAAGTCCCAGTCGAACG 5′

Thus, the sequences coding for the sense and antisense siRNA are separated by a minimal TAR loop coded by the separating sequence SEQ ID No. 1 coding for the minimal sequence recognized by the TAT protein (SEQ ID No. 4).

Coding for the siRNA:
5′ 3′
GCAAGCTGACCCTGAAGTTCATCTGAGCTCTGAACTTCAGGGTCAGCTTG
CCGTTCGACTGGGACTTCAAGTAGACTCGAGACTTGAAGTCCCAGTCGAA
CG

### EXAMPLE 2

### Inhibition of the Expression of GFP by the siRNAGFP Expressed by the Vector of Example 1

Mammalian cells COS-7 are transfected with polyfect (Qiagen) with 4 μg of expression vectors of the siRNA (pTATOF-siRNAGFP, part A, or pSuper siRNAGFP without TAR loop, part B) as well as an expression vector of Green Fluorescent Protein or GFP (500 ng). Sixty hours after transfection, a Western blot was performed from the total extracts using an antibody directed against the GFP (Santa cruz) or cellular tubulin (Sigma) in order to evaluate the quantity of proteins used for this test.

The cells are cultivated in a DMEM (Gibco) environment containing 10% of fetal bovine serum in the presence (wt) or absence (−) of a vector for the expression (0.5 μg) of TAT protein or of its mutants, co-transfected (V. Bres et al., Nat. Cell Biol. August 2003; 5(8): 754-61, the subject matter of which is incorporated by reference).

A control is performed with cells that did not receive the expression vector of siRNA (pTATOF-siRNAGFP or pSuper siRNAGFP) cultivated in the absence of TAT protein. Likewise, controls are performed with a vector for the expression of the mutated TAT protein (incapable of bonding the TAR sequence) in the presence of the vector for the expression of siRNA (pTATOF-siRNAGFP or pSuper siRNAGFP).

FIG. 1 shows the results of this experiment:

Part A:

In the absence of TAT protein and of pTATOF-siRNAGFP, the GFP is present in the cells (column 1).

In the absence of TAT protein and in the presence of the of pTATOF-siRNAGFP vector, the GFP is degraded (column 2).

In the presence of wild TAT protein and of the of pTATOF-siRNAGFP vector the GFP is present in the cells (column 3) in a quantity comparable to that present in the cells that received neither TAT protein nor pTATOF-siRNAGFP vector (column 1) and is therefore not degraded.

In the presence of mutated TAT protein and of the of pTATOF-siRNAGFP vector the GFP is degraded in the cells (columns 4, 5).

Part B:

In the absence of TAT protein and of pSupersiRNAGFP vector (without TAR sequence) the GFP is present in the cells (column 1).

In the absence of TAT protein and in the presence of the pSupersiRNAGFP vector the GFP is degraded (column 2).

In the presence of wild or mutated TAT protein and of the pSupersiRNAGFP the GFP is degraded in the cells (columns 1, 4 and 5).

This demonstrates that the TAT protein recognizes the separating sequence intercalated between the sense and antisense sequences of siRNA and blocks its activity when a mutated TAT protein incapable of recognizing the separating sequence has no effect on the siRNA that appears perfectly functional.

1-14. (canceled)

15. A method for inducing activity of an RNAi in cells comprising:
introducing TAT protein and a nucleic acid coding for sense and antisense sequences of an RNAi of interest into eukaryote cells, which sense and antisense sequences are separated by a nucleotide separating sequence comprising at least SEQ ID No. 1, coding for at least one nucleotide sequence contained in a TAR sequence for a time sufficient to be recognized by the TAT protein under conditions in which the nucleic acid is transcribed in RNA so that the transcribed separating sequence and the TAT protein form a complex inhibiting the activity of the RNAi of interest; and
withdrawing the TAT protein to induce acitivity of the RNA.

16. The method according to claim 15, wherein the nucleic acid comprising the sequences coding for the sense and antisense sequences of an RNAi of interest separated by the separating sequence is introduced into the cell in the form of a vector.

17. The method according to claim 16, wherein the vector is a plasmid or a viral vector.

18. The method according to claim 16, wherein the nucleic acid is under the control of a transcription promoter.

19. The method according to claim 16, wherein the nucleic acid further comprises an antibiotic resistance gene.

20. The method according to claim 19, wherein the antibiotic resistance gene is a neomycin resistance gene.

21. The method according to claim 15, wherein transfected cells are mammalian cells.

22. The method according to claim 15, wherein the TAT protein is introduced into the eukaryote cells by cultures of the cells in a culture environment containing the TAT protein.

23. The method according to claim 22, wherein transcription of the RNAi is induced by cultivating the eukaryote cells in an environment that does not contain TAT protein.

24. The method according to claim 15, wherein the TAT protein is introduced into the eukaryote cells by introducing an inducible vector comprising a nucleotide sequence coding for the TAT protein into the cells.

25. The method according to claim 24, wherein transcription of the RNAi is induced by blocking synthesis of the TAT protein.

26. A cell or a line of cells transfected by the nucleic acid according to claim 15.

27. A pharmaceutical composition comprising at least one nucleic acid according to claim 15 and, optionally, a compatible excipient.

28. A pharmaceutical composition comprising a cell or line of cells according to claim 26 and, optionally, a compatible excipient.


SEQUENCE LISTING (5 sequences)

SEQ ID NO: 1 - DNA, 11 bp, Human immunodeficiency virus
atctgagctct

SEQ ID NO: 2 - DNA, 20 bp, Artificial Sequence
  Description of Artificial Sequence Synthetic oligonucleotide
gcaagctgaccctgaagttc

SEQ ID NO: 3 - DNA, 20 bp, Artificial Sequence
  Description of Artificial Sequence Synthetic oligonucleotide
gaacttcagggtcagcttgc

SEQ ID NO: 4 - DNA, 51 bp, Artificial Sequence
  Description of Artificial Sequence Synthetic oligonucleotide
gcaagctgaccctgaagttcatctgagctctgaacttcagggtcagcttgc

SEQ ID NO: 5 - RNA, 11 nt, Human immunodeficiency virus
aucugagcucu